(19) United States
(12) Patent Application Publication
(10) Pub. No.: US 2008/0234750 A1
(43) Pub. Date: Sep. 25, 2008

(54) Anterior vertebral plate with taper lock screw

(76) Inventors: Richard W. Woods, Catonsville, MD (US); Todd W. Wallenstein, Ashburn, VA (US)

Correspondence Address: PERRY E. VAN OVER & ASSOCIATES, PLLC, P.O. BOX 399, FAIRFAX, VA, 22038, US

(21) Appl. No.: 11/700,234

(22) Filed: Jan. 31, 2007

(51) Int. Cl. A61B 17/70; A61B 17/58; A61B 17/04

(52) U.S. Cl. 606/291; 606/280; 606/301

(57) ABSTRACT

Provided is a novel system that includes a low profile anterior vertebral body plate and taper lock screws for the fixation and stabilization of the cervical spine, the anterior vertebral plate having a novel screw locking mechanism attached to the screw during the manufacturing thereof and providing a taper lock fit with the anterior vertebral plate. Also provided is a method of stabilizing cervical vertebrae using the disclosed device.

### BACKGROUND OF THE INVENTION

1. Technical Field

The present invention relates to devices and methods for use in orthopedic spine surgery. In particular, the present invention relates to a system that provides a low profile anterior vertebral body plate and taper lock screws for the fixation and stabilization of the cervical spine, the anterior vertebral plate having a novel screw locking mechanism attached to the screw during the manufacturing thereof and providing a taper lock fit with the anterior vertebral plate.

2. Background Art

Disease, the effects of aging, or physical trauma resulting in damage to the spine has been treated in many instances by fixation or stabilization of the effected vertebra. The use of plates and screws for fixation and stabilization of the cervical vertebra has been widely accepted as a reliable practice and has proven to be highly successful clinically.

The various plates, which are attached to the anterior vertebral bodies of the spinal column by bone screws have some common features such as relatively planar body profiles that define multiple holes or slots through which the screws fit and are threaded into the bone. Various means have been used to prevent the screws from becoming loose or detached from their necessary secured or locked attachment to the vertebral plate. Among the differences between the conventionally used plates and screws is the manner in which the screws are locked into place in the hole or slot of the plate after the screws have been secured to the bone.

These conventional devices can be generally grouped into three basic categories with regard to how the screws are captured or secured in the plates.

Early plate designs were standard bone plates having holes through which screws were passed and screwed into the bone. These plates had no special provision for attaching the screws to the plate and as such were susceptible to having the screws back out of the plate over time. There have been clinically reported instances of screws backing out of these type plates with resulting surgical complications. Due to the potential and actual unreliable performance of such plates, the need for secure fixation of the screw to the plate as well as to the bone is now considered a basic requirement for vertebral plates. Due to the lack of predictable security of the screw to the plate, plates which do not secure the screw relative to the plate have fallen out of favor and virtually disappeared from use.

Efforts have been made to secure the screws relative to the plates. In one design the screw head contains a threaded hole configured to receive a set screw. After the screw has been driven into bone and the head is seated in the plate hole, the set screw is inserted into the receiving hole of the screw head. The set screw is tapered to cause the screw head to expand and frictionally engage the wall of the plate hole, thereby resisting forces which tend to cause the screw to back out. While such mechanisms have worked to some degree, the addition of a small additional part, the set screw, at the time of surgery presents the potential hazard of dropping the set screw into the surgical field or otherwise misapplying the set screw to the screw head, for example, cross threading.

An alternative approach has been to provide features in the plate, which are specifically designed to hold the screw in position once the screw is inserted through the plate and screwed into the bone. One direction taken in this effort has been to design plates that incorporate or attach individual retaining rings or snap features associated with each plate hole configured to hold the inserted screw in place relative to the plate. These plates are very common and widely used; however, an inherent problem associated with such plates is the use of the additional very small retaining elements that can become disengaged from the plate and migrate into the surrounding soft tissues. Further, difficulty experienced in accessing and disengaging the small locking elements and removing the screws from this type of plate has caused some concern for the continued use of such plates. A similar approach involves individual cams associated with each plate hole, which when rotated apply friction pressure to the screw head in an attempt to resist back out.

Another approach is to add a cover to the plate after the screws have been placed. Such a design undesirably adds steps to the surgical procedure, thickness or height to the overall construct, and is susceptible to misapplication. Yet another direction taken in this effort to provide plates with locking elements is to provide dedicated overlying features, which are attached to the top side of the vertebral plate for the purpose of covering at least a portion of the screw head and thereby holding the screw in a seated and locked position. Generally these plates are designed to provide a variety of screw covering features that are pre-attached to the plate, and which can be selectively slid or rotated into position once it has been inserted. In some devices, such covering plates cover multiple screw heads. These plates typically require an increase in the overall composite thickness of the plate in order to accommodate the additional locking feature attached to the top side of the plate. This is a particularly unacceptable condition due to the use of such plates in an area of the spine where a thin, smooth surfaced profile for the plate assembly is preferred. Another major problem with such plates is that the overlying locking element cannot always be properly positioned over the screw head if the screw shaft was, due to anatomical necessity, positioned through the plate and into the bone at an angle such that the screw head does not fully seat in the plate recess provided on the top side of the plate. Further, when one of the overlying locking elements of such a plate loosens or becomes disengaged it is then free to float away from the top side of the plate and migrate into the soft tissue adjacent to the top side of the vertebral plate.

Yet another approach is to provide machine threads in the plate hole with corresponding threads on the screw head. Thus the screw has a first, bone engaging thread on its shaft and a second machine thread on the screw head. As the threaded shaft is screwed into bone the screw head approaches the plate hole and the machine thread engages the thread in the hole. Aside from the fact that there is nothing to prevent the same forces that urge the screw to back out of bone to have the same effect on the machine thread engagement, such an arrangement does not provide adequate clinical flexibility. First there is no assurance that the lead in thread of the machine thread will match up with the plate hole thread when the screw head reaches the hole, raising the possibility of cross threading. Second, the machine thread in the plate hole does not allow various angular positions between the screw and the plate, as the threads must match up and engage when the screw head reaches the hole. As to the latter point, one plate provides a threaded ring in the plate hole, which is intended to allow the head to assume a variety of angular positions.

There is therefore, an unfulfilled need for an anterior cervical plate system that can maintain a relatively low profile, as found in the non-locking plates while providing the security of a locking plate system and doing so no matter how angulated the inserted screw may be relative to the plate. Further there is a need for a vertebral plate that does not have locking elements with the predictable problems of locking elements becoming disengaged from the plate and migrating away from the top side of the plate into the surrounding soft tissue.

### SUMMARY OF THE DISCLOSURE

The present invention meets the above identified need by providing a low profile anterior vertebral body plate, which is secured to the underlying bone using novel taper lock screws.

Also provided is a low profile anterior vertebral body plate, which is secured to the underlying bone using novel taper lock screws having screw heads with circular or convex shaped lateral surfaces that correspond to the shape of the concavity of a circumferentially disposed tapered locking ring.

Also provided is a low profile anterior vertebral body plate, which is secured to the underlying bone using novel taper lock screws, each of the screw heads being able to rotate within a respective tapered locking ring prior to the screw and locking ring being moved into a seated and locked position in the plate.

Also provided is a low profile anterior vertebral body plate, which is secured to the underlying bone using novel taper lock screws, the screws being pre-assembled with a tapered locking ring.

Also provided is a low profile anterior vertebral body plate, which is secured to the underlying bone using novel taper lock screws, the screws being pre-assembled with a tapered locking ring, the circumference of the locking ring being interrupted by a relief slot that permits limited expansion of the internal diameter of the tapered locking ring during pre-assembly with the screw head and also permitting limited compression of the internal diameter of the tapered locking ring as the tapered locking ring is fully engaged with the correspondingly tapered hole in the plate such that when fully seated in the hole, the taper locking ring securely locks the screw into position within the plate.

Also provided is a method of stabilizing spinal vertebrae, the method including providing a low profile anterior vertebral body plate, which is securely attached to the underlying bone of adjacent vertebrae using novel taper lock screws so as to hold one attached vertebra in a fixed position relative to the adjacent attached vertebra.

Also provided is a kit, which includes at least one low profile anterior vertebral body plate and a corresponding set of novel taper lock screws.

### BRIEF DESCRIPTION OF THE DRAWINGS

The foregoing and other features of the low profile anterior vertebral body plate and novel taper lock screws will become apparent to one skilled in the art to which the device relates upon consideration of the following description of exemplary embodiments with reference to the accompanying drawings, wherein:

FIGS. 1 A-E show respectively a top view, isometric view, end view, side view, and cross-sectional end view of the plate with two taper lock screws fully seated and locked into the holes of the plate.

FIGS. 2A-D show respectively a top, isometric, first side, and alternate side view of the screw with tapered locking ring assembled. FIG. 2C shows a side view of the assembled screw and tapered locking ring with the relief slot showing on the tapered locking ring.

FIGS. 2E-F show respectively a side view and isometric view of the screw and the tapered locking ring prior to assembly of the two components.

FIGS. 3A-D show respectively a first side view, isometric view, an alternative side view, and a bottom view of the bone screw prior to assembly with the tapered locking ring component.

FIGS. 4A-D show respectively a top view, isometric view, first side view with the relief slot showing, and an alternative side view of the tapered locking ring prior to assembly with a screw head.

FIGS. 5A-E show respectively a top view, isometric view, end view, side view, and cross-sectional view of the low profile anterior vertebral body plate with multiple holes for receiving respective taper lock bone screws.

### DETAILED DESCRIPTION OF PREFERRED EMBODIMENTS

Detailed embodiments of the present invention are disclosed herein; however, it is understood that the following description and each of the accompanying figures are provided as being exemplary of the invention, which may be embodied in various forms without departing from the scope of the claimed invention. Thus, the specific structural and functional details provided in the following description are non-limiting, but serve merely as a basis for the invention as defined by the claims provided herewith. The device described below can be modified as needed to conform to further development and improvement of materials without departing from the inventor's concept of the invention as claimed.

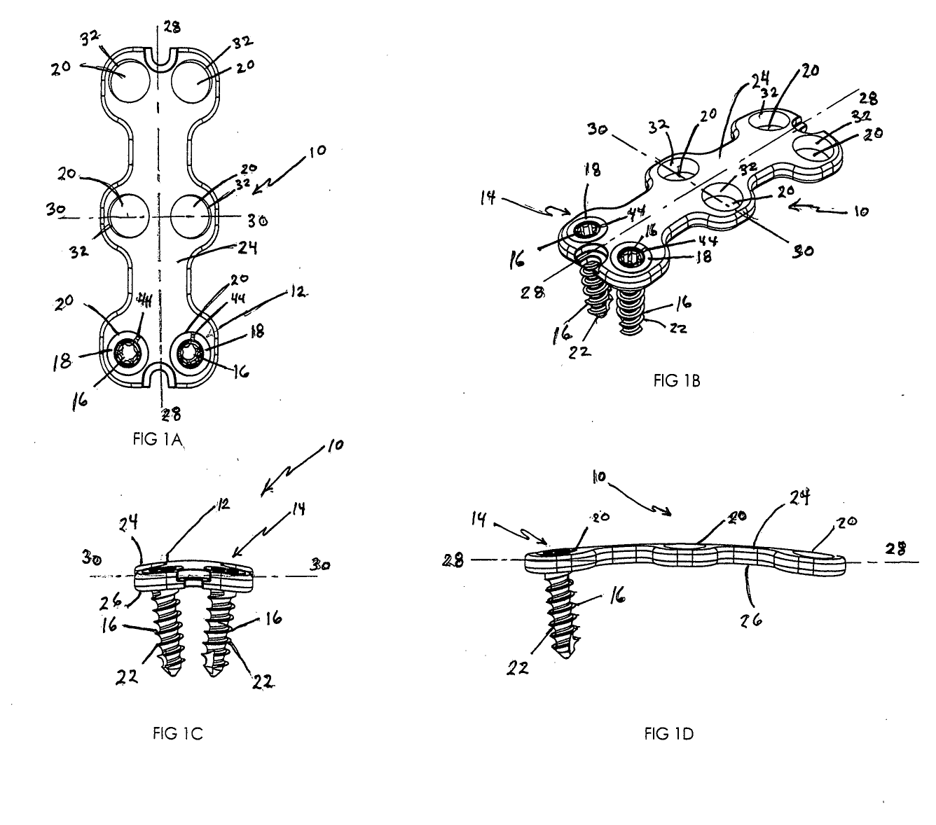

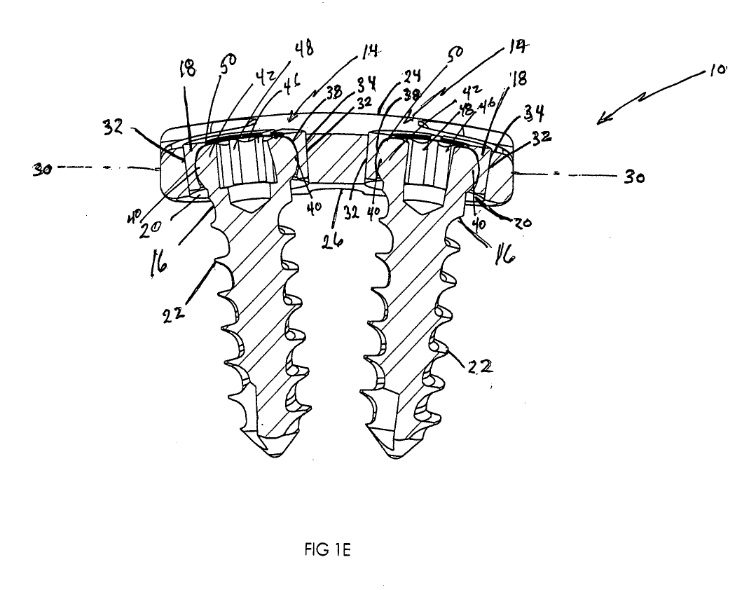

The device, as generally shown at 10 in FIG's 1A-E is a low profile anterior vertebral body plate 12 that, when implanted in a patient can be secured to the underlying bone using novel taper lock screw assemblies, which are generally shown at 14 in FIGS. 1A-E and 2A-F and include a threaded bone screw 16 and a tapered locking ring 18. The vertebral body plate 12, as best shown in FIGS. 1A-D and 5A-E can be provided as an elongated, low profile, plate structure that defines at least one and preferably multiple tapered screw holes 20, which provide through passage for the threaded portion 22 of the threaded bone screw 16 from the plate upper surface 24 to the plate lower surface 26.

As shown in FIGS. 1A-E and 5A-E, the plate 12 can be configured to be generally planar; however, the plate preferably will be formed to have arcuate upper and lower surfaces 24, 26, arcing along both the longitudinal axis 28 as well as the transverse axis 30 of the plate 12. This arcing of the plate surface provides a better conformational fit to the anterior surface of the vertebrae to which the plate is to be attached. Each of the screw holes 20, which are defined as through passages in the plate 12, is provided with a tapered side wall 32. The degree of inward taper from upper to lower portion of the screw hole side wall 32 corresponds to the degree of inward taper from upper to lower portion of the outer wall 34 of the tapered locking ring 18. Preferably, the taper is a Morse type taper; however other types of taper can be used without departing from the scope of the invention.

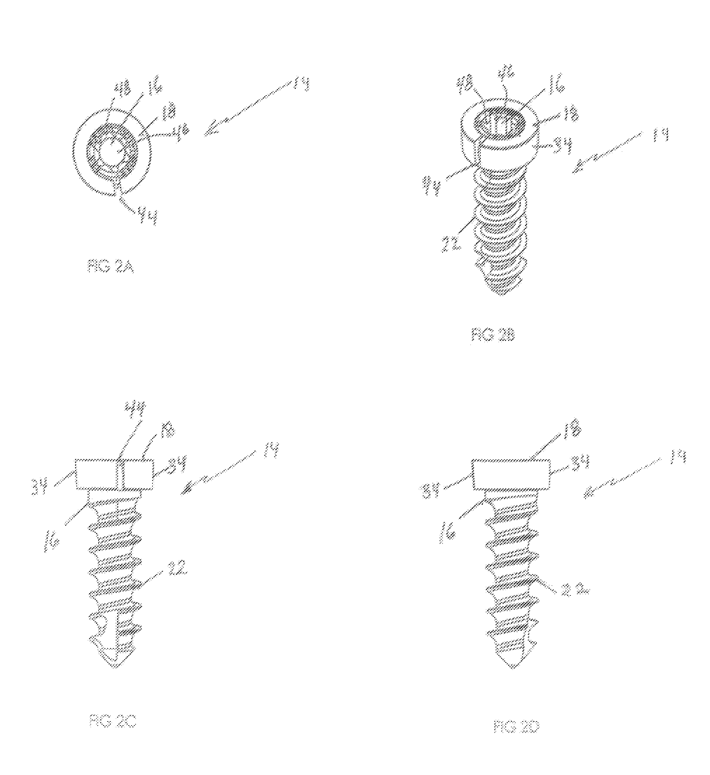

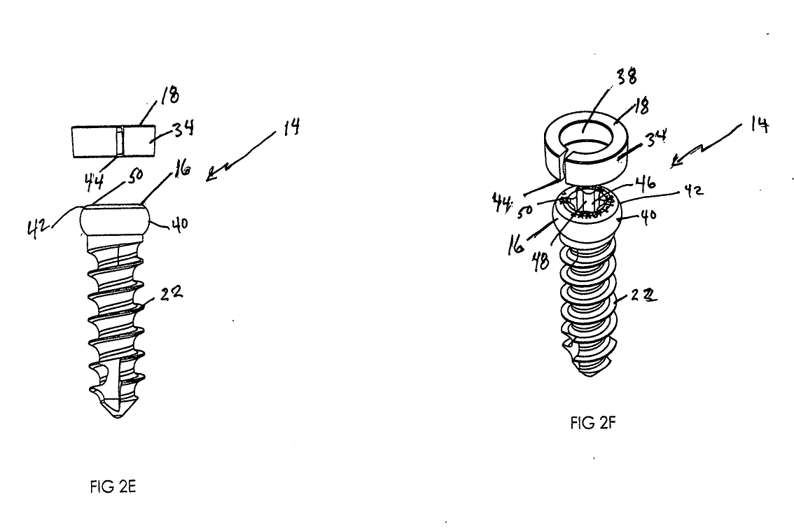

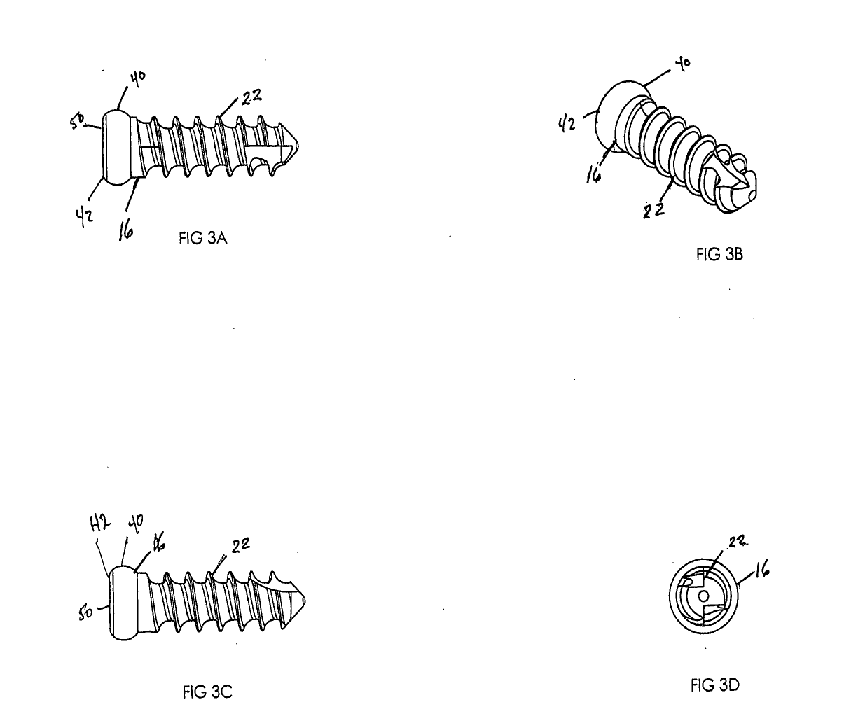

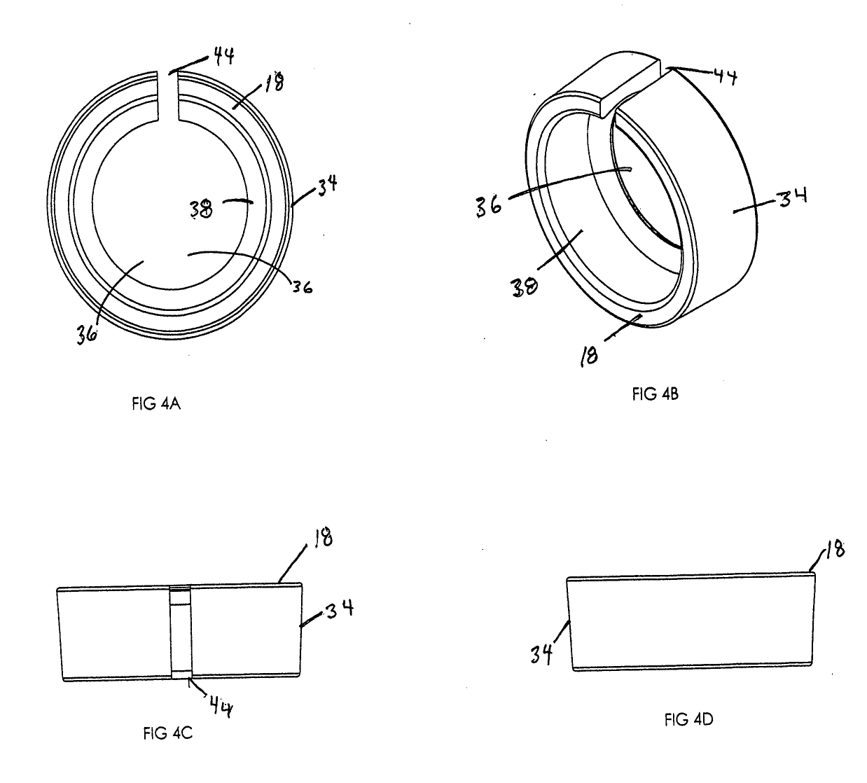

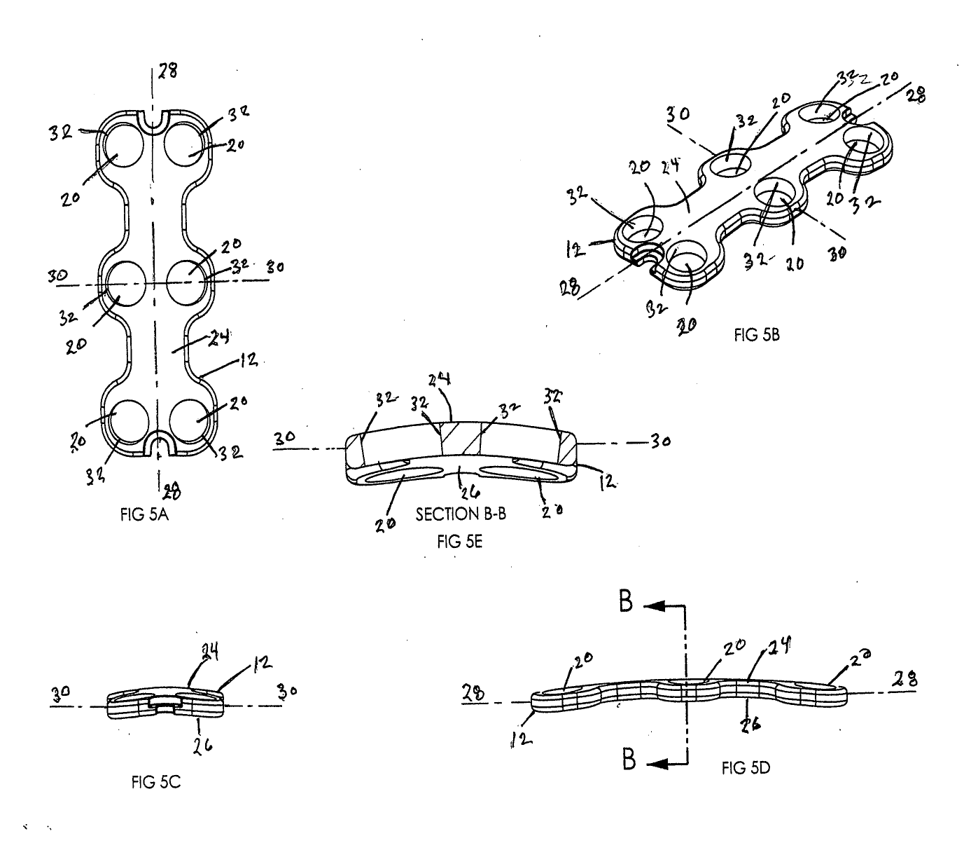

The tapered locking ring 18 defines a through lumen 36, which is formed to have a generally concave shaped inner wall 38 that is sized and configured to rotatably receive and hold the complimentary convex shaped outer side wall 40 of the head 42 of the bone screw 16. As shown in FIGS. 2A, B, C, E, and F, the tapered locking ring 18 is provided with a expansion/compression relief slot 44, which serves to break the circumferential continuity of the tapered locking ring 18 such that if compressive forces are exerted inward about the circumference of the locking ring 18, the relief slot 44 can decrease in size so as to enable the locking ring 18 to absorb those compressive forces and decrease in diameter, albeit doing so with an outward bias to return to the original larger dimensioned normal configuration. Similarly, if expansive forces are exerted outward against the concave shaped inner wall 38 of the tapered locking ring 18, the relief slot 44 can accommodate those expansive forces and allow an increase in diameter of the locking ring 18, albeit with an inward bias to return to the original smaller dimensioned normal configuration.

The flexibility provided by the relief slot 44 is important to the function of assembling of the screw 16 to the tapered locking ring 18 to form the preassembled taper lock screw assembly 14. The convex shaped outer wall 40 of the screw head 42 is sized and configured to be capable of being preassembled into the concavity formed by the inner wall 38 of the tapered locking ring 18. This preassembly is easily achieved by forcing the convex shaped outer wall 40 of the screw head 42 into the concavity of the inner wall 38 of the tapered locking ring 18. The joining and fit of the two components is a snap fit relationship in that the expansive forces created by the forcing of the screw head 42 into the concavity of the locking ring lumen 36 is absorbed by the relief slot 44 until the screw head 42 is in place within the locking ring 18, at which time the locking ring yields to its bias to return to its normal smaller diameter size and configuration. Once preassembly of the taper lock screw assembly 14 is completed, the convex surface of the screw head 44 is free to rotate within the concavity of the locking ring 18 but is restrained from separating from within the locking ring lumen 36 due to the normal size of the locking ring lumen openings, which are sufficiently smaller than the diameter of the screw head 44. This preassembly of the taper lock screw assembly 14 makes it possible in practice to insert the screw through the screw hole 20 of the plate 12 into the underlying bone and then lock the screw 16 into place without the need to attach and manipulate small additional locking elements or components as is commonly required with conventional screw locking plate systems.

The flexibility provided by the relief slot 44 is also important to the function of locking the taper lock screw assembly 14 into position within the plate 12. As shown in FIG. 1E, the rotational relationship of the convex shaped screw head 40 with the concave shaped inner wall of the locking ring 38 allows the screw to be inserted into the bone through the screw hole 20 of the plate 12 at virtually any angle necessary. This polyaxial feature of the taper lock screw head assembly 14 in relation to the plane of the plate 12 is a tremendous advantage to providing the best possible connection to the bone. As shown in FIGS. 1A, B, and E and FIGS. 2A, B, and F, a tool receptacle 46 having tool gripping elements 48 can be defined in the upper surface 50 of the screw head 40. The tool gripping elements can be of any configuration that is suitable for facilitating the gripping of the screw head by a correspondingly configured tightening and/or loosening tool. As the preassembled taper lock screw assembly 14 is rotated inward by the action of a tightening tool, the screw threads 16 engage the underlying bone drawing the taper lock screw assembly 14 down into a sliding engagement with the screw hole tapered side wall 32. As the tapered locking ring 18 slidably engages the tapered side wall 32 of the screw hole 20, the locking ring 18 is forced to move into the screw hole 20 with an alignment coincident with the taper of the hole 20. This alignment of the tapered surfaces of the assembly 14 with the screw hole 20 necessarily causes the convex shaped screw head 40 to rotationally adjust within the concavity of the tapered locking ring 18 so as to accommodate the already well established axis of entry of the threaded portion 22 of the screw 16 in the bone. Thus, the taper lock screw assembly 14 interaction with the tapered surface of the screw hole 20 provides the polyaxial feature of the device 10. As the screw 16 continues to be driven into the underlying bone, locking ring tapered outer wall 34 continues to engage and finally friction locks to the tapered side wall 32 of the screw hole 20. This friction locking engagement exerts radial compressive force on the tapered locking ring 18, which at least partially closes or narrows the normal space of the relief slot 44 thereby decreasing the diameter of the tapered locking ring and the space within the concavity of the locking ring lumen 36. These compressive forces are transferred to the convex shaped screw head 42 so as to hold and lock the screw head 42 in position relative to the plate 12.

Thus, the device 10 as described herein advantageously permits the screw 16 to be inserted into bone at a variety of angles relative to the plane of the plate, for example, polyaxial insertion, and with continued insertion of the screw 16 into bone, taper lock screw assembly 14 locks the screw into position relative to the plate 12.

The foregoing method of use of the device 10 can be employed as a method of stabilizing or fixing an injured or diseased vertebra and if necessary, multiple devices or a device, which is elongated beyond the examples depicted herein, can be employed as necessary. A reversal of rotational torque on the screw head using a tool designed to generate sufficient torque to overcome the taper lock established between the taper lock screw assembly 14 and the plate 12 can serve to remove the screw from the plate and thus remove the plate from a patient if necessary. The amount of force necessary to overcome the taper lock is greater than that required to simply unscrew the threaded portion 22 of the screw 16 from the bone underlying the plate and is also greater than commonly experienced micro-motion or other forces which can act to cause a conventional screw to back out of the bone.

While the device as described herein can be preferably used to attach to the anterior surface of cervical vertebrae and is configured to be capable of stabilizing cervical vertebrae, it is within the inventors' understanding that the plate can be configured and adapted to conform to any implantable surgical plate requirement to provide a low profile plate capable of securing and stabilizing any injured or diseased bone.

The device 10 can be manufactured as integral components by methods known in the art, to include, for example, molding, casting, forming or extruding, and machining processes. The components can be manufactured using materials having sufficient strength, resiliency and biocompatibility as is well known in the art for such devices. By way of example only, suitable materials can include implant grade metallic materials, such as titanium, cobalt chromium alloys, stainless steel, or other suitable materials for this purpose. It is also conceivable that some components of the device can be made from plastics, composite materials, and the like.

It is also within the concept of the inventors to provide a kit, which includes at least one of the vertebral plate and taper lock screw systems disclosed herein. The kit can also include additional orthopedic devices and instruments; such as for example, instruments for tightening or loosening the bone screws, spinal rods, hooks or links and any additional instruments or tools associated therewith. Such a kit can be provided with sterile packaging to facilitate opening and immediate use in an operating room.

Each of the embodiments described above are provided for illustrative purposes only and it is within the concept of the present invention to include modifications and varying configurations without departing from the scope of the invention that is limited only by the claims included herewith.

What is claimed is:

1. A novel bone plate system, comprising:
A plate having an upper surface and a lower surface, said plate defining at least two bone screw holes, said bone screw holes having tapered inner surfaces with said taper decreasing from said upper surface to said lower surface of said plate,
a taper lock screw assembly for each of said at least two bone screw holes, said taper lock screw assembly comprising a threaded bone screw having a head portion, the surface of at least a side portion of said head portion being curvate or convex shaped and a threaded portion suitable for threaded entry into bone,
said taper lock screw assembly also comprising a tapered locking ring having an external tapered surface corresponding to said taper surface of said bone screw holes and a concave inner lumen complimentary in size and shape to the convex portion of said screw head, such that said screw head can be rotatably fitted within said lumen of said tapered locking ring, said tapered locking ring being provided with a relief slot passing through the entirety of the wall of said tapered locking ring,
wherein said bone screw head can be locked into position relative to said plate by compressive forces against said tapered locking ring when said taper lock screw assembly is fully seated within said bone screw hole.

2. The bone plate system of claim 1, wherein said bone screw is capable of polyaxial alignment with said bone screw hole.

3. The bone plate system of claim 1, wherein said device is an anterior vertebral body plate.

4. The bone plate system of claim 1, wherein said bone screw and said tapered locking ring are preassembled to provide said taper lock screw assembly prior to use in a surgical procedure.

5. The bone plate system of claim 1, wherein said plate is configured to have an upper and lower curved surface, said curve being along the longitudinal axis of the plate.

6. The bone plate system of claim 1, wherein said plate is configured to have an upper and lower curved surface, said curve being along the transverse axis of the plate.

7. The bone plate system of claim 5, wherein said curve is also along the transverse axis of the plate.

8. The bone plate system of claim 1, having a low profile such that no features of said device extend above the level of the upper surface of said plate.

9. The bone plate system of claim 1, wherein said bone screw head and said tapered locking ring are capable initially of rotational interaction such that said bone screw when fully seated and locked into position within said plate is capable of being polyaxial relative to said bone screw hole.

10. The bone plate system of claim 1, wherein said taper of said bone screw hole and said corresponding taper of said taper lock screw assembly is a Morse type taper.

11. The bone plate system of claim 4, wherein said preassembly of said taper lock screw assembly is a snap fit assembly wherein said relief slot in said tapered locking ring is capable of facilitating said snap fit assembly.

12. The bone plate system of claim 1, wherein said relief slot in said tapered locking ring is capable of facilitating transfer of said compressive forces of said tapered locking ring against said bone screw head so as to lock said bone screw head into position relative to said plate.

13. A method of stabilizing a vertebral body, the method comprising,
providing a bone plate system according to claim 1,
surgically accessing an anterior surface of a vertebral body in need of stabilization,
positioning said bone plate and attaching same to said vertebral body using said taper lock screw assemblies.

14. A kit comprising at least one system according to claim 1 and at least one other tool or instrument for use in orthopedic surgery.
